(19) United States
(12) Patent Application Publication
(10) Pub. No.: US 2021/0307664 A1
(43) Pub. Date: Oct. 7, 2021

(54) Biometric Mood Sensing Apparatus and Method of Use

(71) Applicant: Lamont I. Bey, Pittsburgh, PA (US)

(72) Inventor: Lamont I. Bey, Pittsburgh, PA (US)

(21) Appl. No.: 17/222,591

(22) Filed: Apr. 5, 2021

Related U.S. Application Data
(60) Provisional application No. 63/005,038, filed on Apr. 3, 2020.

(51) Int. Cl. A61B 5/16; A61B 5/00; A61B 5/021

(52) U.S. Cl. CPC A61B 5/165; A61B 5/742; A61B 2560/04; A61B 5/021; A61B 5/681; A61B 5/7405

(57) ABSTRACT

A biometric mood sensing bracelet comprises: a bracelet body having an outer face, an inner face, a distal edge and a proximal edge, said inner face including a plurality of sensors for measuring a plurality of biometrics from a wrist region of a wearer of the bracelet body; a plurality of batteries contained within the bracelet body; a plurality of LED's coupled to an exterior of the bracelet body, said plurality of LED's forming a capable of displaying one or more of the measured plurality of biometrics; and a microprocessor coupled to the bracelet body, said microprocessor being in operational communication with the plurality of sensors to: (a) translate the measured plurality of biometrics into a predominant mood of the wearer of the bracelet body and (b) send to the plurality of LED's a color indicative of the predominant mood translated from the wearer's measured plurality of biometrics.

### CROSS-REFERENCE TO RELATED APPLICATIONS

This application is a perfection of pending U.S. Provisional Application Ser. No. 63/005,038, filed on Apr. 3, 2020, the disclosure of which is fully incorporated herein.

### STATEMENT REGARDING FEDERALLY SPONSORED RESEARCH OR DEVELOPMENT

Not Applicable.

### THE NAMES OF THE PARTIES TO A JOINT RESEARCH AGREEMENT

Not Applicable.

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not Applicable.

### STATEMENT REGARDING PRIOR DISCLOSURES BY THE INVENTOR OR A JOINT INVENTOR

Not Applicable.

### BACKGROUND OF THE INVENTION

### (1) Field of the Invention

The disclosure relates to biometric wearable devices. More particularly, it relates to a new biometric device or apparatus for making a user aware of his or her emotions in real time and the effects of these emotions on the wearer's body for getting the user/wearer of such devices more aware/in synch with their measurable moods and overall health.

### (2) Description of Related Art Including Information Disclosed Under 37 CFR 1.97 and 1.98

The prior art relates to biometric wearable devices. Existing devices track biometrics and display them on a smart phone/watch screen and/or transmit them to a personal electronic device. These known devices are typically employed for exercising, e.g., steps/miles walked and general health. But they don't make the user/wearer readily aware of representative mood changes that can be instantaneously interpreted from a plurality of device measured biometric data. This invention also adds other sensory indicators including sounds (bells, bings, whistles, etc.), lit screen messages (action recommendations), vibrations and/or temperature changes (going from cool to warm or vice versa) along with the aforementioned mood color change indications.

Other patented, or patent pending references include, but are not limited to: U.S. Pat. Nos. 9,974,481, 9,907,473, 9,893,818, 9,737,212, 9,288,836, 9,173,052, 9,028,405, 7,441,415, 6,675,744, and 6,619,835; U.S. Published Application Nos. 20160183795, 20140206955, 20120130196, 20060235330 and 200100123588; European Application No. EP 3,041,200, WIPO Application No. WO 2016/037276, and Chinese Patent No. CN 2015/10298639.

One proposed name for this product is HEALTHLOCK™ wrist bands.

### BRIEF SUMMARY OF THE INVENTION

One embodiment of this disclosure comprises a moderately wide bracelet body between about 2-4 inches across but is not so thick as to be intrusive. That bracelet body has an outer face, an inner face, a distal edge and a proximal edge. The bracelet's inner face is sufficiently spaced from a user/wearer's wrist so as define a body cavity between. Some embodiments of this bracelet body are rather inflexible and configured to be affixed to a user/wearer's wrist nearest his/her hand. An alternate variation would employ new or existing materials for providing such biometric wrist band displays with a limited degree of flexibility for both installation onto a wrist and day-to-day wearing. To a lesser extent, it may be possible for one or more of these wrist band displays to be replaced with even smaller wristband straps or retractable band components.

A plurality of sensors couple to the bottom portion of this bracelet body interior. They are intended to extend through the bracelet's inner face and measure, in real time, the bracelet wearer's heart rate and blood pressure along with still other biometrics like blood sugar (or glucose) levels, oxygen absorption measurements and/or cholesterol. Still other measurements may include bone densities, readings on vital organ performance and/or current hydration levels.

Ideally, versions of this device may be able to make wireless contacts with such medical professionals so as to promptly alert them (and perhaps also emergency personnel—i.e., paramedics and EMS) of a detected medical condition that warrants more immediate attention due to pulse rates, blood pressure drops/increases, arrhythmia, and/or the bodily detections of food allergy reactions, the presence of high alcohol contents and possibly even the presence of certain drug levels (legal, prescription or illegal).

In yet another variation, this bracelet may include a full body display/diagram for the wearer to make note of where he/she may be experiencing pain/discomfort or other perceived ailments for scheduling a consult with medical professionals to test and/or discuss later.

A plurality of batteries and LED's will couple to this bracelet body component as seen in the cross-sectional view at FIG. 8. In one view, a curved display screen couples to the top exterior of this bracelet body. Preferably, this curved display is at least about 270 degrees around the wearer's wrist. Lesser preferred variations may extend a full 360 degrees about the wearer's wrist depending on the availability of making display screens more flexible. To a lesser extent, a “bracelet” with an interior of body sensors can be resized for positioning around other body parts including but not limited to a wearer's upper arm, ankle, etc. to measure/body levels/vital signs from other monitoring areas.

A microprocessor is coupled to the main bracelet body within the body cavity. It stays in operational communication with the sensors, the plurality of batteries, the plurality of LED's and a display screen, if separate from the LED's. This microprocessor takes spaced/timed readings or continuous monitoring from the sensors, and correlates biometrics from these sensors with one of several mood/emotions, before illuminating the plurality of LED's in one solid color that correlates to the emotion(s) detected.

This invention represents an improvement over today's smart watch monitoring technologies. It is meant to measure the wearer's moods biometrically—e.g., anger, joy, fear, etc. Once a particular mood has been measured biometrically, the visual display would change from its view of mottled, mixed colors—swirling about, to one solid color indicative of the mood duly detected. For instance, a solid red banding color would form about most if not all of the bracelet's exterior (outer band surround) in the calculation of anger detection. When used to further calculate/correlate heart rates and blood pressure levels (among other biometrics), the device of this invention could also vibrate, flash warnings/colors and/or sound audible alarms. Yet another variation could visually warm the bracelet wearer with preventive measures to be taken . . . imminently. For example, a word message WM, like “BREATHE”, or eat a candy bar for blood sugar levels, sit/rest could flash across the top bracelet display area(s).

There has been outlined, rather broadly, the more important features of this invention. Yet still other features (some optional) will be described hereafter that form the subject matter of the appended claims.

The inspiration for the invention was to create a better way for people to understand how their emotions affect their bodies. This invention enables its wearer to stay more in tune with his/her emotional state.

### Benefits

The device increases a wearer's awareness of their own emotional state and how such emotions may affect the body as a whole. The device offers assistance with how to recognize then cope with emotional and behavioral issues.

It may help to prevent negative outcomes in a person's day-to-day experiences.

It may help to promote emotional and physical well being.

It is easy to wear and use.

It has a unique stylistic design.

It is ideal for individuals with stress disorders and/or weight issues.

### Features

It consists of a wearable electronic device with EM sensing technologies designed to detect various low-level molecular resonances in the wearer's body for determining and displaying emotional status.

The metal bracelet can be made from aluminum alloys, lithium cobalt oxide, carbon graphite, gold, copper, silver, etc.

The circuit boards would feature a round design—about 1 cm thick.

These electromagnetic sensors would collect biometric data (heart rate, blood pressure, etc.), at least periodically and possibly continuously in some instances. The bracelet would then use microprocessor-based analyses to generate a color-based display of the wearer's emotional state.

Additional features include a haptic engine (vibration), ambient light sensor, heart rate display, blood pressure display, time display and a screensaver.

Once the device senses the mood, the mood description will appear within the corresponding color and the alleviation message will display on the unit.

### Marketing:

drug stores and pharmacies

electronic stores

online retailers

wholesalers of medical supplies.

Related names: Potential trademarks for of branding of this invention include: E-MO™; Embrace your emotions; and Wear Your EMOtions on the wearer's wrist.

To a lesser degree, this device can be wirelessly linked to coordinate with the wearer's other existing electronic device(s) including but not limited to smart phones, iPads, PC's, laptops . . . even a smart watch worn on the wearer's other wrist.

### BRIEF DESCRIPTION OF THE DRAWING(S)

The disclosure will be better understood when consideration is given to the following detailed description made with reference to the accompanying drawings in which:

FIG. 1A is a front perspective view of one embodiment of this invention looking axially into a closed bracelet 10 that displays representative biometrics (i.e. body vitals D) onto a predominantly black background;

FIG. 1B is an upper right perspective view of the same bracelet 10 from FIG. 1A;

FIG. 2 is a top plan view of the bracelet from FIGS. 1A and B as worn on the right wrist WW, between his/her hand WH and forearm WF for displaying vital wearer data D;

FIG. 3 is a front axial perspective view of another embodiment of wrist bracelet per this invention showing its “at rest”, screensaver display of various emotion colors on screen as would be seen, in motion;

FIG. 4A is a front axial perspective view of a first variation of “at rest” screensaver on a black screened wrist bracelet showing a digital display of current time in an array of numbers made from moving emotion colors. FIG. 4A also shows a wrist bracelet interior having a plurality of body sensors embedded in a padded inner wrist wrap;

FIG. 4B is a front axial perspective view of a second variation of “at rest” screensaver on a moving colored background MC onto which would be displayed the current time T in white numbered digits. Optionally, current day/date displays (not shown) may supplement the time T display;

FIG. 5 is a front axial perspective view of two representative displays D (a value for heart rate over the most recently measured blood pressure values). Once an emotional state of the wearer has been “determined”, the previously mentioned blend of moving colors MC will form into one solid color indicative of the wearer's most dominant emotion ascertained by a microprocessor MP calculation from the biometrics measured;

FIG. 6 is a front axial perspective view of a second displayed color emotion, e.g., red representing “anger” when worn on a wearer/user's wrist. For emotions like anger, fear, anxiety or anything else that elevates the wearer's blood pressure and/or pulse rate, preferred embodiments may further warn said wearer by mildly vibrating on the wrist as indicated by curved lines V. It may further visually notify the wearer by flashing a word message WM, and also audibly beeping, to stop a minute and take a much-needed breath (or “breathe”) for calming down;

FIG. 7A is a front axial perspective view showing one version of bracelet with a plurality of sensors S, said bracelet having an attachment segment 22 on a rotatable hinge RH for fitting about the wrist of its wearer/user;

FIG. 7B is a front axial perspective view showing a second rotatable hinge RH segment 22 for reconnecting via a physical hook latch J to an appropriately sized and positioned eyelet E after inserting the wearer/user's wrist inside;

FIG. 7C is a front axial perspective view showing a third version of spring ball SB, on a rotatable hinge RH section 22 that connects to a duly sized indentation or ball recess BR;

FIG. 8 is a cross-sectional view of the one embodiment of bracelet 10 having alternating sections of body sensors S connected to a microprocessor MP with batteries 24 and padding for wearer comfort about the bracelet's inner face, along with an exterior visual display made from a plurality of interconnecting LED's 26 extending almost fully about the bracelet's exterior (circumference);

FIG. 9 is a block diagram of one embodiment showing main component parts of this invention and

FIG. 10 is a top plan view showing the various color codes-to-emotions that may be (temporarily) displayed, and possibly visually indexed, per another preferred embodiment.

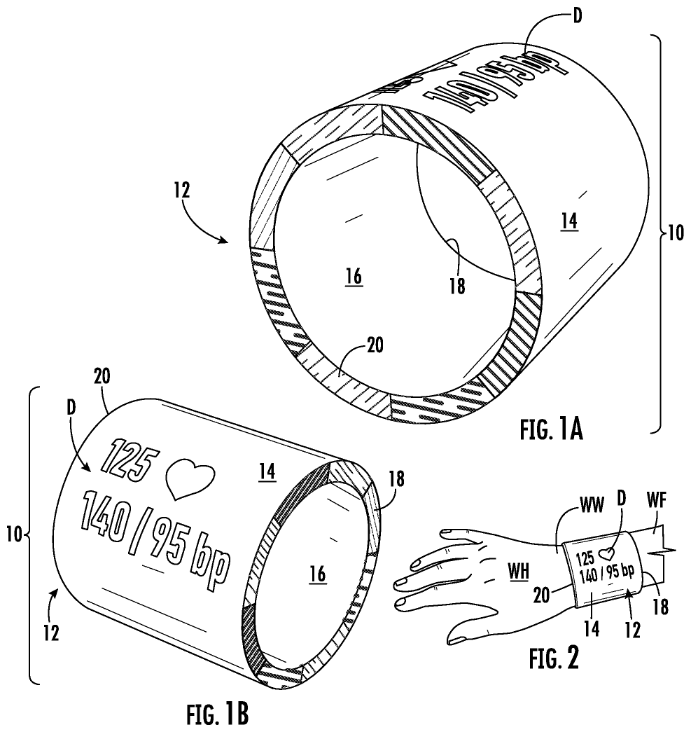

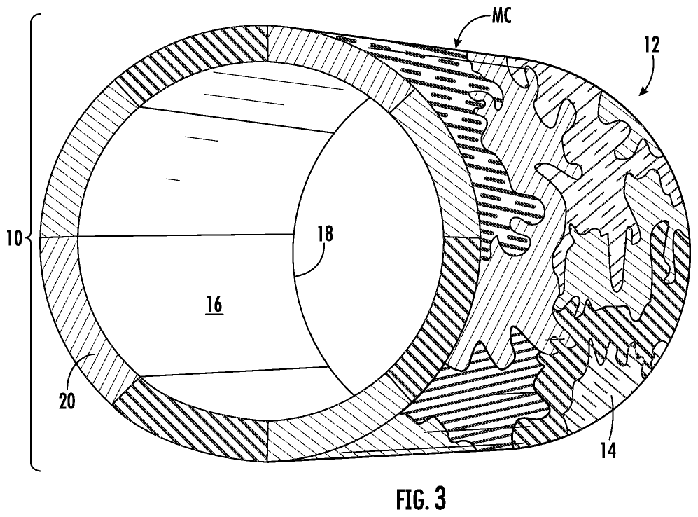

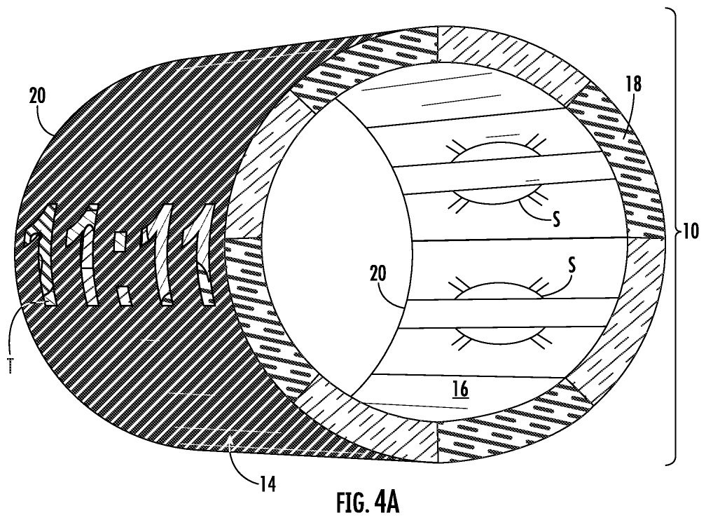

### DETAILED DESCRIPTION OF THE INVENTION

The new biometric, mood sensing bracelet/apparatus of this invention, generally 10, comprises a bracelet body 12 having an outer face 14, an inner face 16, a distal edge 18 and a proximal edge 20. Outer face 14 is translucent and sufficiently spaced from the bracelet's inner face 16 so as to define a body cavity region (not shown) about the bracelet wearer's wrist. Preferably, outer face 14 is made from a scratch-resistant, glare-resistant (preferably polymer) material. Bracelet body 12 should be somewhat flexible and configured for wearing about a user's wrist WW closest to his/her hand WH.

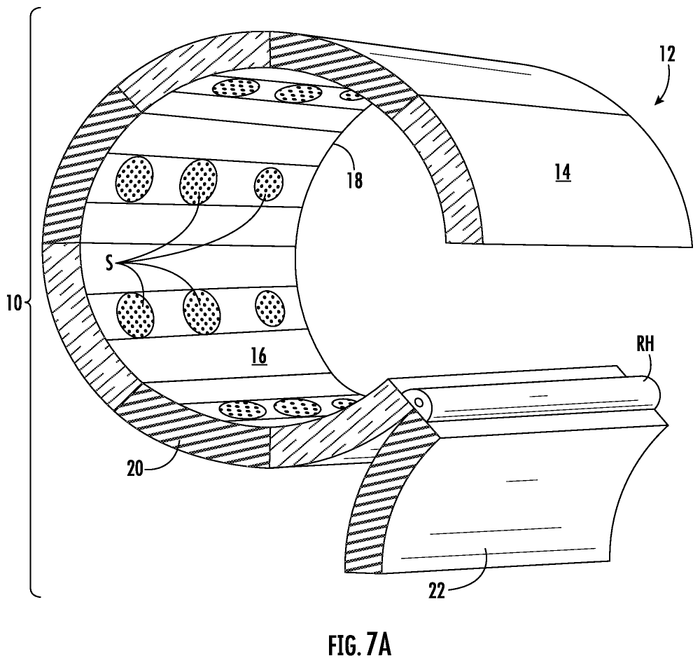

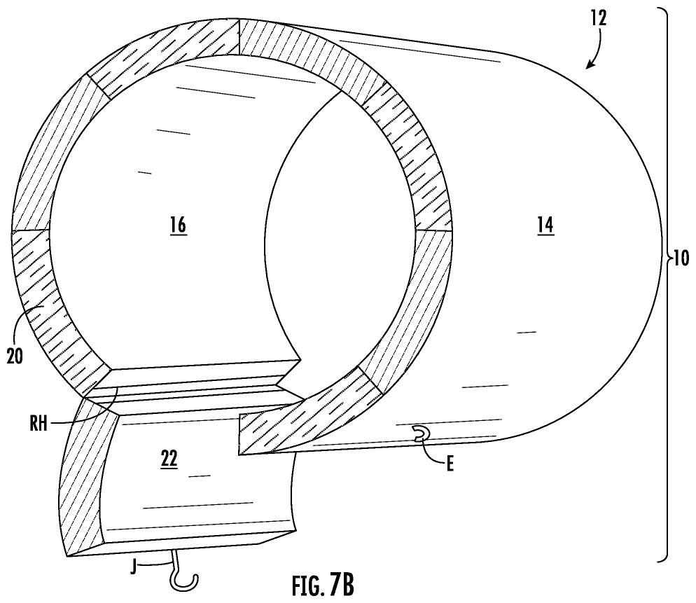

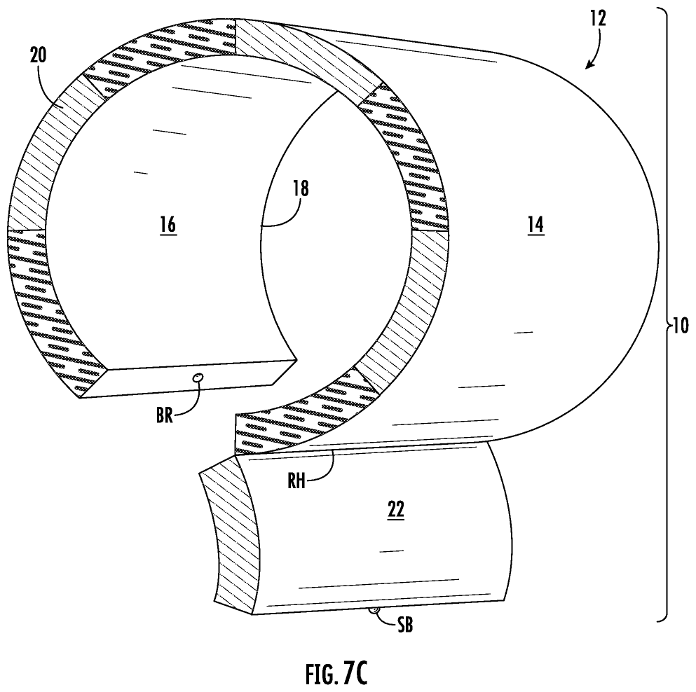

Bracelet body 12 may include a latch/clasp or other selectively engage-able mechanism such as, but not limited to, a hook-and-loop fastener, a buckle or the like. Representative mechanisms are depicted in accompanying FIGS. 7A through C: FIG. 7A showing a rotatable hinge RH; and FIG. 7B a rotatable hook “J” for latching into a corresponding eyelet E on an adjacent section of bracelet body exterior also rotatably hinged RH thereto. FIG. 7C includes a spring-loaded button SB for partnering with a button recess BR on an open internal edge of hinged section 22. Alternately, the bracelet body as a whole may be made with an elastic insert for expanding and then fully sliding onto the wearer's wrist.

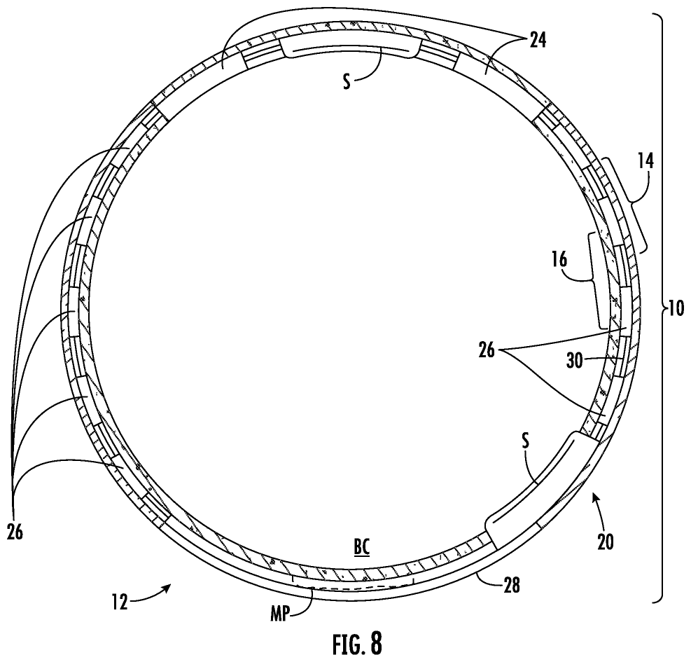

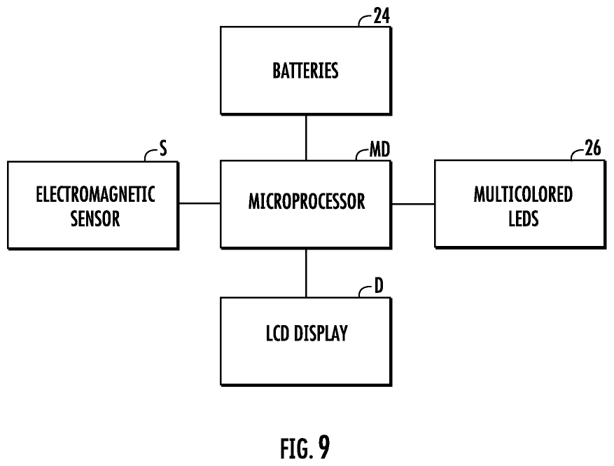

With respect to FIG. 8, one or more internal sensors S couple to the inner face 16 of bracelet body 12. Preferably, such sensors would at least partially extend through one or more sections/slices of that inner face 16 for making at least periodic (if not, continuous) contact with the user's wrist. Such sensors would detect that user's heart rate and blood pressure among other biometric measurements. Sensors S may be electromagnetic or employ other known or subsequently developed vitals-measuring materials/means.

A plurality of batteries 24 couple inside the main bracelet body 12 for powering up the unit. These batteries may be accessed through sliding covers in either the unit's outer face 14, inner face 16 or via one lateral edge (either distal 18 or proximal 20). These batteries 24 can be made for periodic removal and replacement, or for occasional recharging from an external power source.

Also in FIG. 8, there is graphically depicted a plurality of LED's 26 that may interconnect to one another for forming a continuous, curved display screen about all or most the bracelet's outer face 14. There may be five or more LED's 26 within each of the left and right portions/halves of the main bracelet body 12. The resulting curved display screen may be flush with outer face 14 and should be flexible to some extent for improved user comfort.

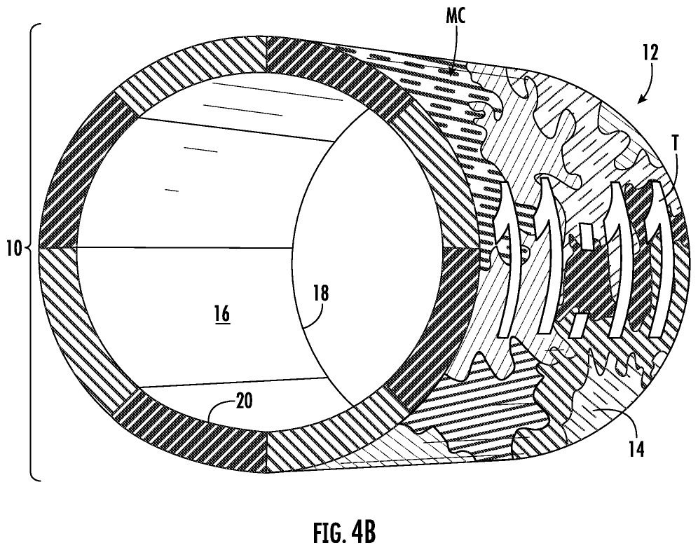

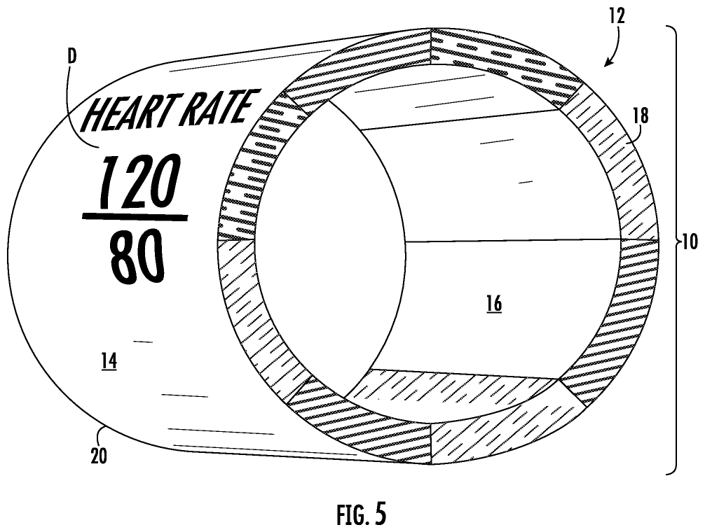

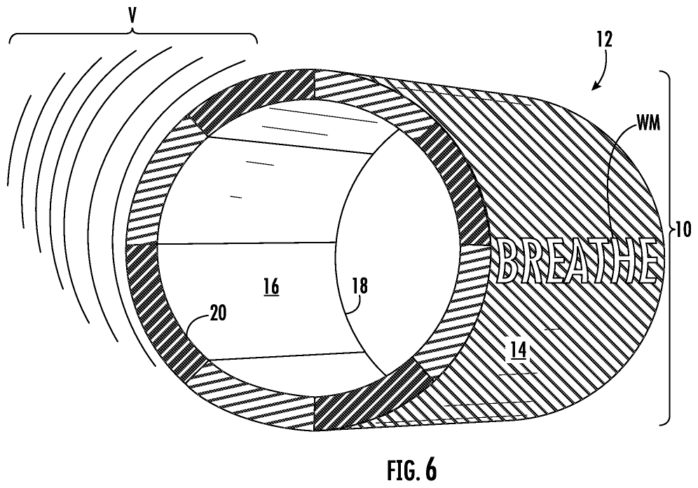

A microprocessor MP may be contained within the intermediate layers of bracelet body 12, yet operationally connect to the plurality of sensors S extending about inner face 16, the power batteries 24 therefor and the plurality of LED's 26. This microprocessor MP takes readings from these sensors S and correlates the measured biometrics into one or more wearer emotions/moods before illuminating a color code for said emotion/mood onto the plurality of LED screens 26. In a “rest state”, these LED's may display mottled, preferably moving combinations of color splotches before being converted into the single solid color display indicative of the bracelet wearer's emotion/mood. When “at rest”, the display may default to show current time T with FIGS. 4A and B show alternate visual displays therefor.

Microprocessor MP shows numerically measured biometric readings (from sensors S) on the plurality of LED displays 26—and may further include one or more corresponding wearer messages WM for alerting the user of a potentially vulnerable medical condition requiring imminent attention. Representative display messages may include advice for the user to calm down, “take a deep breath” or “go for a walk”. The actual biometric data D readings include, but are not limited to, heart rate and blood pressure and numerous others mentioned hereinabove.

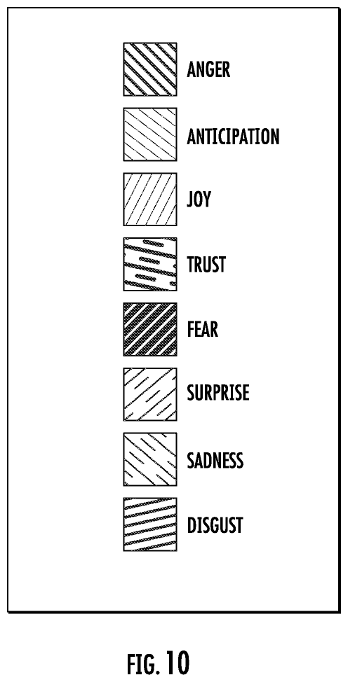

In use, the bracelet body may be worn continuously on its wearer/user's wrist. The wearer/user may glance down periodically to check on actually measured and displayed biometric data D. Or, the wearer/user may rely on visual translations of such biometric data into perceived emotion/mood detections that would get color-displayed on a bulk of the bracelet exterior screens. FIG. 10 shows some of the possible color code-to-emotion translations made possible with one version of this invention.

With respect to the above description, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the invention.

Therefore, the foregoing is considered as illustrative only of the principles of disclosure. Further, since numerous modifications and changes will readily occur to those skilled in the art, it is not desired to limit the disclosure to the exact construction and operation shown and described, and accordingly all suitable modifications and equivalents may be resorted to, falling within the scope of the disclosure.

### SEQUENCE LISTING

Not applicable

Having described the best modes currently known for practicing this axle replacement system and method, it is to be understood that the scope of this invention may be further described by the attached claims.

What is claimed is:

1. A biometric mood sensing bracelet comprising:
a bracelet body having an outer face, an inner face, a distal edge and a proximal edge, said inner face including a plurality of sensors for measuring a plurality of biometrics from a wrist region of a wearer of the bracelet body;
a plurality of batteries contained within the bracelet body;
a plurality of LED's coupled to an exterior of the bracelet body, said plurality of LED's forming a capable of displaying one or more of the measured plurality of biometrics; and
a microprocessor coupled to the bracelet body, said microprocessor being in operational communication with the plurality of sensors to: (a) translate the measured plurality of biometrics into a predominant mood of the wearer of the bracelet body and (b) send to the plurality of LED's a color indicative of the predominant mood translated from the wearer's measured plurality of biometrics.

2. The biometric bracelet of claim 1 wherein the plurality of LED's are capable of numerically displaying one or more of the wearer's measured plurality of biometrics.

3. The biometric bracelet of claim 1 wherein the plurality of LED's are capable of displaying a plurality of mottled colors until being converted into the color indicative of the predominant mood translated from the wearer's measured plurality of biometrics.

4. The biometric bracelet of claim 3 wherein the plurality of mottled colors randomly move about the plurality of LED's.

5. The biometric bracelet of claim 1, which is made from a flexible material.

6. The biometric bracelet of claim 1 wherein the plurality of batteries are replaceable.

7. The biometric bracelet of claim 1 wherein the plurality of batteries are rechargeable.

8. The biometric bracelet of claim 1 wherein the plurality of measured biometrics include heart rate and blood pressure.

9. The biometric bracelet of claim 1, which further includes a vibration alarm component.

10. The biometric bracelet of claim 1, which further includes a sound alarm component.

11. The biometric bracelet of claim 1, which further includes a temperature changing alarm component.

12. The biometric bracelet of claim 1, which further includes means for displaying a written message of one or more words across the plurality of LED's as a visual alarm.

13. The biometric bracelet of claim 1, which further includes a current time display component.

14. The biometric bracelet of claim 1, which further includes means for measuring a blood sugar level of the wearer.

15. The biometric bracelet of claim 1, which includes at least one hinged segment for opening and closing about the wrist of the wearer.

16. The biometric bracelet of claim 1, which is at least about 2 inches in total length.

17. The biometric bracelet of claim 1, which is about 4 inches or less in total length.

18. The biometric bracelet of claim 1 wherein the color indicative of the predominant mood translated is a single solid color.

19. A method of using a biometric mood sensing bracelet to indicate a sensed mood of the bracelet's wearer, said sensed mood being translated from a plurality of biometric data measured by the mood sensing bracelet, said method comprising:
(a) providing a bracelet having: (i) a main body having an outer face, an inner face, a distal edge and a proximal edge, said inner face including a plurality of sensors for measuring a plurality of biometrics from a wrist region of the bracelet's wearer; (ii) a plurality of batteries contained within the main body; (iii) a plurality of LED's coupled to an exterior of the main body, said plurality of LED's forming a capable of displaying one or more of the measured plurality of biometrics; and (iv) a microprocessor coupled to the main body, said microprocessor being in operational communication with the plurality of sensors to: (a) translate the measured plurality of biometrics into a predominant mood of the wearer of the bracelet and (b) send to the plurality of LED's a color indicative of the predominant mood translated from the wearer's measured plurality of biometrics;
(b) positioning the mood sensing bracelet on a wearer's wrist; and
(c) activating the display screen on the mood sensing bracelet.

20. The method of claim 15 wherein the mood sensing bracelet further includes at least one of: a vibration component, a sound alarm component, a temperature emitting component and a visual word display component.
